(19) United States
(12) Patent Application Publication
(10) Pub. No.: US 2024/0409872 A1
(43) Pub. Date: Dec. 12, 2024

(54) PHOTOBIOREACTOR METHODS AND SYSTEMS FOR ACCURATE ENVIRONMENTAL MAINTENANCE OF BIOLOGICAL CULTURES

(71) Applicant: COLORADO SCHOOL OF MINES, Golden, CO (US)

(72) Inventors: Devin KARNS, Seattle, WA (US); Edward DEMPSEY, Golden, CO (US)

(21) Appl. No.: 18/739,057

(22) Filed: Jun. 10, 2024

Related U.S. Application Data
(60) Provisional application No. 63/506,996, filed on Jun. 8, 2023.

(51) Int. Cl. C12M 1/34; C12M 1/00; C12M 1/06

(52) U.S. Cl. CPC C12M 41/26; C12M 23/38; C12M 27/02; C12M 29/06; C12M 41/06; C12M 41/12; C12M 41/44

(57) ABSTRACT

Methods and systems for controlling microorganism culture conditions are disclosed. The system may comprise a sample vessel and plumbing free metal parts, and use a modular cap design to adapt to a multitude of analytical probes, and possess a square profile with non-reflective surfaces. The system may also include stirrer; a lighting control module, a lighting element may produce stable light intensity range; a temperature control module that may be located at the bottom of the sample vessel; a pH control; a gas sparging control module; a dilution control module; a liquid storage system; an electronic modularity; a weighing module; and a scheduling system. In many embodiments of the system and method, the pH control may hold the pH within 0.1 of a target pH, the weighing module may monitor volume, and the scheduling system may allow the creation of user defined events and set points.

### CROSS REFERENCE TO RELATED APPLICATIONS

This application claims the benefit of priority to U.S. provisional application No. 63/506,996, filed Jun. 8, 2023, which is incorporated by reference herein in its entirety and for all purposes.

### FIELD

The present inventive concept is related to the precise control of photobioreactor conditions to emulate environments such as outdoor algal ponds.

### BACKGROUND

During study of small or microorganisms in solutions, controlling conditions and parameters of the solution and environment is often helpful to capture scientifically significant or repeatable data. Accordingly, these studies are often performed in lab or artificial environments due to the unpredictability of conditions in natural bodies of water. However, generating conditions in labs that reliably emulate natural conditions is often very difficult if not impossible. Accordingly, there is a need for an artificial environment capable of reliably emulating natural conditions.

### SUMMARY

Embodiments of the present invention are directed to a system configured to control microorganism culture conditions is provided, wherein the system comprises: a sample vessel, wherein the sample vessel provides a liquid path free of contact with metal parts, uses a modular cap design to adapt to a multitude of analytical probes, has a square profile, uses a non-reflective surface, and uses a magnetic stirrer; a lighting control module, wherein a lighting element produces stable light intensity range; a temperature control module, wherein the control is located at the bottom of the sample vessel; a pH control, wherein the pH control allows the pH to be held within 0.1 pH of the target pH; a gas sparging control module; a dilution control module; a liquid storage system; an electronic modularity; a weighing module, wherein the weighing module monitors volume; and a scheduling system, wherein the scheduling system may allow the creation of user defined events and set points for all environmental parameters and liquid manipulations.

Also provided is an in-vitro method for culturing a microorganism in a photobioreactor, the method comprising: using a sample vessel with flat sides to emulate pond surface voxel; creating a schedule to control the pH, temperature, liquid volume, dilution, gas sparging, and illumination of the sample vessel; and maintaining the schedule to create a sample vessel environment similar to outdoor ponds.

Also provided is a system for emulating a natural environment in-vitro, comprising: a main enclosure comprising: a lighting control module including a light source, a pH control system, plumbing defining a pathway between inlet valving and outlet valving, one or more sensors selected from one or more of: a pH sensor, a temperature sensor, a mass sensor, a light intensity sensor, a pressure sensor, volume sensor, optical density sensor, or a flow sensor, a growth station platform configured to receive a growth chamber and comprising: a weighing module to determine a mass of the growth chamber, and a stir plate configured to stir a growth chamber, and a temperature control to cool one or both of the main enclosure and the growth chamber. In some embodiments, the system may comprise a growth chamber defining an internal volume and having a non-reflective surface and configured to retain a culture solution; and a cap defining a plurality of connections and selectively connected to a top of the growth chamber to selectively seal the internal volume; wherein at least one of the one or more sensors is coupled to the cap and captures information about the culture solution, and the growth chamber may have a rectangular cross section corresponding to a voxel of a natural body of water, and/or the plumbing may be operatively connected with one or more inlet solution containers and the growth chamber at the cap, and the inlet solution containers include one or more of media for the culture solution, dilution solution, a rinsing solution, or a cleaning solution. In many embodiments, the the light source may be positioned at a top of the growth chamber to direct light vertically through the growth chamber, and/or the pH control system may include a sparger to release a gas through the culture solution to raise or lower the pH value of the culture solution, and/or the gas may be selected from one or more of carbon dioxide to decrease the pH value or air to increase pH value. In some embodiments, the one or more sensors of the system may include an optical density sensor and/or the plumbing may define an at least mostly metal-free pathway for solutions of the system and/or include one or more peristaltic pumps to limit contamination of a solution of the system. In many embodiments, the temperature control may include a thermoelectric cooler positioned at the growth chamber platform and/or include a cooling system for the light source to limit radiant heat transmission from the light source towards the growth chamber platform. In some embodiments, the stackable electronics board assembly may include at least one processing element, and wherein the at least one processing element generates or executes a schedule to control one or more of a pH, temperature, liquid volume, dilution, or light intensity of the culture vessel and/or the at least one processing element is in operative communication with a remote device to selectively update a schedule or communicate information captured by the one or more sensors of the culture solution.

Also disclosed is a system configured to control microorganism culture conditions comprising: a culture vessel including a culture solution, wherein: the culture vessel provides a liquid path at least partially free of contact with metal parts, includes a modular cap designed to selectively connect with analytical probes or sensors, the culture vessel has a square profile and defines a volume between 100 mL and one liter, the culture vessel defines an at least partially non-reflective interior surface, and the culture vessel includes a magnetic stirrer; a lighting control module, including a lighting element selectively producing a stable light intensity range; a temperature control module including a thermoelectric cooler positioned at a bottom of the culture vessel; a pH control module operatively associated with a gas sparging control module to control the pH of the culture solution; a liquid storage system including storage for one or more of: input media, input dilution solution, input cleaning solution, waste solution, sampling solution; an electronic system configured to execute a scheduling system, wherein the scheduling system defines events and set points for environmental parameters and liquid manipulations; and a weighing module, wherein a mass of the culture vessel is associated with a volume of the culture solution.

Also disclosed are methods for culturing a microorganism comprising: filling a culture vessel having a non-reflective surface with a culture solution to emulate a pond environment under natural conditions; operatively coupling the culture vessel to one or more of a weigh module, temperature control module, light source, plumbing, or a pH control module; executing by a processing element, a schedule to control one or more of a pH, temperature, liquid volume, dilution, or light intensity of the culture vessel; and maintaining the schedule to define culture vessel environment similar to outdoor ponds. In some embodiments, the maintaining step may include; determining a pH of the culture solution by one or more sensors; comparing the determined pH to a desired pH value; and sparging one of air or carbon dioxide within the culture solution to increase or decrease the determined pH to approximate the desired pH value of the culture solution, and/or the maintaining step may include determining a mass of the culture vessel by one or more sensors, wherein the mass is associated with a volume of the culture solution, and adding an inlet solution to the culture vessel by the plumbing thereby increasing the volume of the culture solution, and/or removing a volume of the culture solution for sampling or waste; and adding an inlet solution to the culture vessel by the plumbing to substantially replace the removed volume. In some embodiments, the schedule may define the light intensity within the culture vessel for a period of a day, the light intensity corresponding to a specific geographical location, and wherein the maintaining includes: determining by one or more sensors a temperature of the culture solution, and cooling the culture vessel by a thermoelectric cooler to adjust the temperature to a temperature corresponding to the light intensity and geographical location.

A number of feature refinements and additional features are applicable in the first aspect and contemplated in light of the present disclosure. These feature refinements and additional features may be used individually or in any combination. As such, each of the following features that will be discussed may be, but are not required to be, used with any other feature combination of the first aspect.

In addition to the exemplary aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 depicts a perspective view of an example single station photobioreactor;

FIG. 2 depicts a schematic diagram of an example of the photobioreactor;

FIG. 3A depicts a portion of the main enclosure of the photobioreactor;

FIG. 3B depicts a portion of the main enclosure of the photobioreactor;

FIG. 4A depicts a portion of the growth chamber platform and a portion of the weigh module;

FIG. 4B depicts a portion of the growth chamber platform and a portion of the weigh module;

FIG. 4C depicts a portion of the growth chamber platform and a portion of the weigh module;

FIG. 4D depicts a portion of the growth chamber platform and including the stir plate and temperature control;

FIG. 4E depicts a partial front perspective view of the example bioreactor and growth chamber;

FIG. 5 depicts an example schematic diagram of the photobioreactor;

FIG. 6 depicts a plot of measured and scheduled temperature results during testing of diurnal time courses;

FIG. 7 depicts a plot of measured values of conditions of the bioreactor during diurnal light and temperature with hourly 40 mL dilutions and de-ionized (DI) water replenishments;

FIG. 8 depicts a plot of measured values of conditions of the bioreactor showing nominal set point variance due to temperature variations;

FIG. 9 depicts a plot of measured values of conditions of the bioreactor during 40 mL dilutions with diurnal temperature control.

FIG. 10 depicts a plot of measured values of conditions of the bioreactor during constant temperature control with single manual dilution;

FIG. 11 depicts a plot of measured values of conditions of the bioreactor during constant temperature with diurnal light conditions.

FIG. 12 depicts a plot of measured values of lighting intensity and volume of the bioreactor during geographic diurnal light with constant temperature, a daily dilution before sunset and evaporative losses replenished every two hours;

FIG. 13 depicts a plot of measured values of culture vessel temperature and current draw of a thermoelectric cooler of the bioreactor during geographic diurnal light with constant temperature, a daily dilution before sunset and evaporative losses replenished every two hours;

FIG. 14A depicts a plot of measured values of conditions of the bioreactor while growing picochlorum at constant temperature, geographic diurnal light, with 60% dilution after sunset, bounded pH controls, and air sparging;

FIG. 14B depicts a plot of additional measured values of conditions of the bioreactor under the conditions of FIG. 14A;

FIG. 15A depicts a plot of measured values of conditions of the bioreactor while growing picochlorum at constant temperature, geographic diurnal light, with 60% dilution after sunset, constant pH levels, and pure carbon dioxide sparging;

FIG. 15B depicts a plot of additional measured values of conditions of the bioreactor under the conditions of FIG. 15A;

FIG. 16A depicts a plot of measured values of conditions of the bioreactor while growing picochlorum similar to FIG. 15A, but under light corresponding to morning;

FIG. 16B depicts a plot of additional measured values of conditions of the bioreactor under the conditions of FIG. 16A;

FIG. 17A depicts a plot of measured values of conditions of the bioreactor while growing picochlorum similar to FIG. 17A, but under light corresponding to evening;

FIG. 17B depicts a plot of additional measured values of conditions of the bioreactor under the conditions of FIG. 17A;

FIG. 18A depicts a plot of measured values of conditions of the bioreactor while growing picochlorum at constant temperature, geographic diurnal light, with 60% dilution after sunset, bounded pH, and sparging with pure carbon dioxide after the pH exceeds a threshold value;

FIG. 18B depicts a plot of additional measured values of conditions of the bioreactor under the conditions of FIG. 18A;

FIG. 19A depicts a plot of measured values of conditions of the bioreactor while growing picochlorum similar to FIG. 18A;

FIG. 19B depicts a plot of additional measured values of conditions of the bioreactor under the conditions of FIG. 19A;

FIG. 20A depicts a plot of measured values of conditions of the bioreactor while growing picochlorum similar to FIG. 18A, but over a single day period;

FIG. 20B depicts a plot of additional measured values of conditions of the bioreactor under the conditions of FIG. 20A; and

FIG. 21 depicts a plot of optical density compared the log ratio of the sample photodiode signal against the reference photodiode signal.

### DETAILED DESCRIPTION

The description that follows includes sample systems, methods, and apparatuses that embody various elements of the present disclosure. However, it should be understood that the described disclosure may be practiced in a variety of forms in addition to those described herein.

Successful growth of or information captured regarding lab-grown algal strains, or other organisms, in conventional bioreactors often fails to translate to outdoor productivity or growth under natural conditions. Accordingly, studies or development of lab grown organism in conventional bioreactors may require modifications in view of natural conditions, wasting time and resources to complete outdoor testing. Described herein is a novel, and selectively automated bioreactor and system comprising modes and sub-systems for control of light, temperature, gas sparging, pH, and dilution, as well as methods of using same. The disclosed systems, devices, and methods are useful in simulating or emulating natural environments such as growth day, or series of days, in a lab setting.

The photobioreactor devices, methods, and systems of the present disclosure may include and/or define environmental controls conducive to emulating pond-like (i.e. outdoor, natural, or non-laboratory) conditions and events. In some embodiments, growth chambers or culture bottles may be used with the disclosed devices, methods, and systems. The culture bottles may be of various sizes and shapes.

In one embodiment, the culture bottles may possess a square profile or shape. In some embodiments, the square culture bottle's profile may act as or help emulate a surface volumetric element of a pond. In these embodiments, the termination of the optical path through the bottle may be a non-reflective surface to reduce back reflections. Temperature may be controlled, in the presently disclosed embodiments, at the bottom of the culture bottle, which may help to minimize reflections from thermal materials and components.

In many embodiments, a metal-free liquids path may be used to help reduce or eliminate impact of corrosive media components. In various embodiments, a weigh module may monitor a weight of a culture solution in the growth chamber, which may be associated with a culture volume. Changes in mass or volume may be addressed by topping off or adding solution to the growth chamber, which may maintain media concentrations. For example, the changes in mass or volume may be due to evaporated fluid, water, or media. These embodiments may aid in maintaining accurate long-term dilutions and culture maintenance. In some embodiments, liquid and electrical connections (e.g. sensors) to the bottle may be made with soft and flexible conduits to minimize impact on volume measurements. For example, the electrical connections may be at a modular cap providing access to the culture solution.

In some embodiments, the system and methods may include software that may allow scheduling of user-defined events or environmental parameters of the growth chamber. These user-defined events may be used to create set points for various parameters, including various environmental parameters and liquid manipulations. In some embodiments, the software allows simulation and application of natural/outdoor/pond-like diurnal days.

In some embodiments, the system may include one or more of a high intensity, liquid cooled light engines (in one case, a 4000K LED with a range of about 20 to about 3500 umol PAR), temperature controllers (in one case, a thermoelectric heater/cooler with about 12 to about 45 C range), mass flow controllers (in some cases, for air and/or CO₂ sparging with up to 1 L/min and 20 mL/min, respectively), pH sensors (for example with control by CO₂ administration and/or gain adjustments for +/−0.1 accuracy, typ.), and liquids controllers (in some examples media in, DI in, additive in, effluent out).

In some embodiments, liquids of the system may be housed in sterile autoclavable containers and administered through substantially or completely metal-free liquids paths. Culture media volume may be monitored, in one example by one or more weigh modules—for long term maintenance of media concentrations and salinity—and/or in some embodiments by pump metering.

Schedule design software may define diurnal light curves with stochastic clouds and pond temperature profiles based on input day and pond parameters, as well as pH and gas control curves and liquid in/out events. Control and monitor parameters may be logged to one or more databases for further quality checking and export. In many embodiments, electronic control of the methods, devices, and systems is modular, which may allow for easy updating with different, modified, and/or additional control and sensing elements.

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims

Reference will now be made to the accompanying drawings, which assist in illustrating various features of the present disclosure. The following description is presented for purposes of illustration and description. Furthermore, the description is not intended to limit the inventive aspects to the forms disclosed herein. Consequently, variations and modifications commensurate with the following teachings, and skill and knowledge of the relevant art, are within the scope of the present inventive aspects.

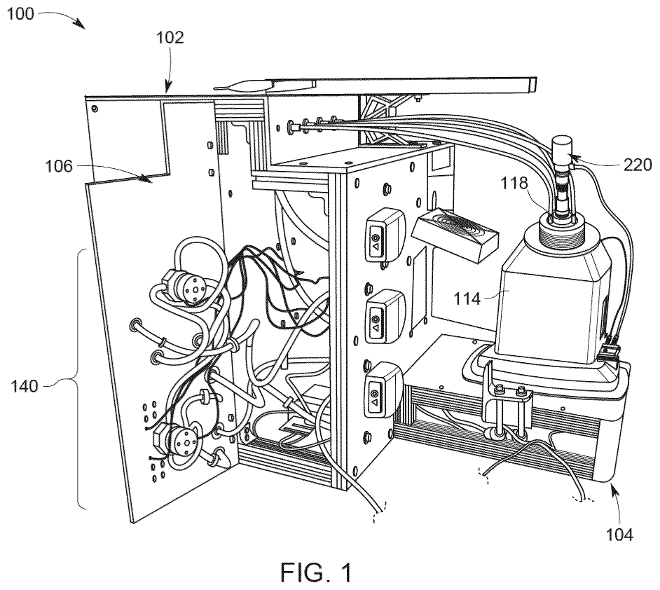

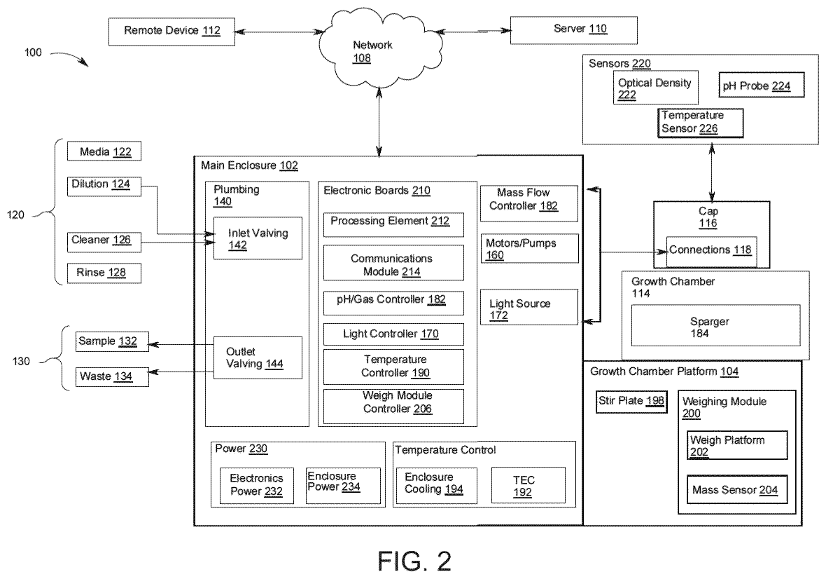

FIGS. 1 and 2 depict an example of a system 100 configured to replicate natural conditions for microorganisms. The system 100 may be a photobioreactor and designed to simulate conditions of natural bodies of water such as ponds in a culture solution. The photobioreactor 100 may emulate liquid solution conditions including solution concentrations, temperatures, pH levels, light intensity, or the like.

The system 100 may include a main enclosure 102 to house the various components, such as in an interior 106. The enclosure 102 may define a growth chamber platform 104 to support a growth chamber or culture vessel 114 in which the culture solution is stored and maintained. The growth chamber platform 104 may include one or more additional components as described herein.

The culture vessel 114 may receive the culture solution for testing or for observing the culture solution over time. The culture bottle may be a modified off the shelf glass sample vessel, which may reduce cost. The culture vessel 114 may have flat sides (e.g. square or rectangular cross section) to emulate or correspond to a pond surface “voxel.” For example, the voxel, or culture vessel 114, may correspond to a three-dimensional portion of a body of water. The culture vessel 114 may include a non-reflective coating or be defined by a non-reflective material. The non-reflective coating or material may limit ingress of unintended external light, reflections within the container, and/or otherwise prevent light conditions different that of a natural environment. The culture vessel 114 includes or contacts components at a bottom or top of the culture vessel 114 to case bottle mounting and/or reduce light reflections or ingress. The culture vessel 114 may have a relatively small volume in comparison to a natural body. For example, the culture vessel 114 may be between 100 mL and 1000 mL, and in one example having a maximum volume of 500 mL.

The culture vessel 114 includes a cap 116 with a modular design. The modular cap 116 design provides adaptability to connect with a multitude of analysis probes, fluid (e.g. liquid or gas) connections, or other components. The modular cap 116 may define or receive ports or fittings. In some examples, the cap 116 defines a plurality of connections 118. In one example, the cap 116 defines 4-port connection to receive or release solutions or gas. In one example, the modular cap 116 included top quick disconnects of stainless steel build, which satisfied most compatibility and function requirements. However, metallic components may show gradual degradation on the sealing surfaces and led to connection reliability problems. Alternatively, cap connections 118, such as liquid connections, may be non-corroding features. In one example, the connections 118 may include SeriesLock Quick Disconnect Fittings from Eldon James, which may be polysulfone quick disconnect-to-barb fittings which feature a spring-free flow path. The connections 118 may be autoclavable. A polymeric or a metal-free path may prevent or reduce corrosion or contamination with liquids in the system 100.

The system or reactor 100 includes plumbing 140 for transferring liquids to or from the growth vessel 114. The system 100 or plumbing 140 may include nearly or mostly metal-free paths. The metal-free paths reduce or eliminate impact of corrosion on parts from saline media, and maintains liquid component integrity for longer periods. The materials of the plumbing, or materials in which liquids may come into contact include one or more of platinum-cured silicone, PVDF, PTFE, glass, chrome-plated stainless steel 316 L, titanium, polypropylene, bioprene, polyethersulfone, PEEK, silicone rubber, or viton. The plumbing 140 may include embedded tubing to reduce or prevent the formation of static charges within metal free path.

The plumbing 140 includes inlet valving 142 for transferring input solutions 120 through the enclosure 102 or to the growth chamber 114. The input solutions 120 may include media 122 for addition to the growth chamber 114. The input solutions 120 may include dilution solution 124 for addition to the growth chamber 114. The input solutions 120 may include a cleaner 126, such as bleach, to sterilize and/or remove solutions and/or contaminants (for example buildup of solution components or bio-material) from the plumbing 140. The plumbing 140, or inlet solutions 120, can include a rinse solution 128 to remove or flush solutions and/or contaminants from the plumbing 140. The plumbing 140 includes outlet valving 130 for transferring effluence outlets 130, such as samples 132 or waste 134, from the enclosure 102 or to the growth chamber 114.

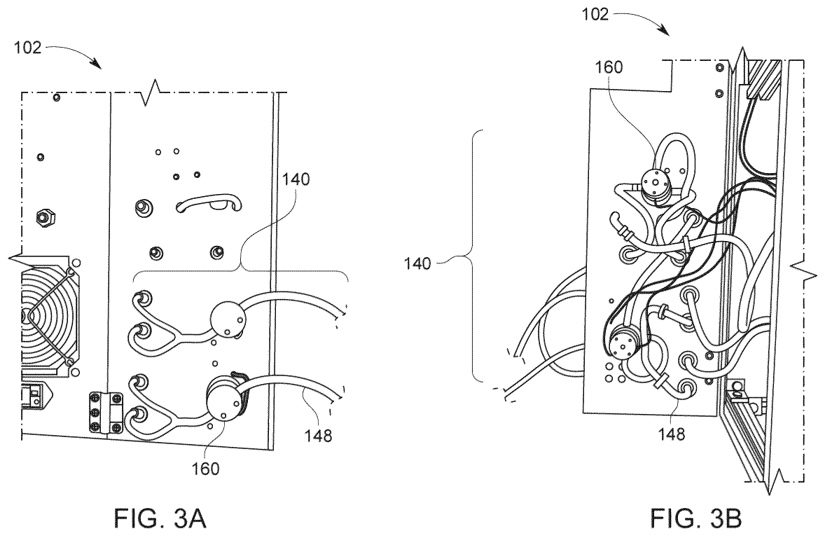

The plumbing 140 includes one or more motors or pumps 160 to dispense liquid through the plumbing. The pumps or motors 160 may be peristaltic pumps, or similar pumps that may avoid direct contact between the liquids and the pump. The peristaltic pumps 160 may also help to prevent or reduce corrosion or contamination. As shown in FIGS. 3A and 3B, the pumps 160 and plumbing 140 may be mounted interior to or external to the main enclosure 102.

In some examples, the plumbing 140 or system 100 includes a liquids corral. The liquid corral may include containers for storing or dispensing media 122 and DI 124 vessels. The plumbing to the vessels may include filters or paths. For example, the plumbing 140 can include such as Polycap filtered inlets and outlets, with filter sizes less than about 1 μm—for example about 0.2 or about 0.1 μm. In many embodiments, the plumbing 140 can include a 0.2 μm filtered vent. The plumbing 140 may include one or more sensors 220 to monitor a pressure, temperature, flow rate, volume, or the like of the various components.

The liquid corral or external plumbing 140 can be connected to or stored in and a stainless steel caddy (not shown), or other autoclavable structure. Filters may mount to the caddy, such as to holding features like inner rungs, hooks clips or the like. Bottles may be placed or connected to the caddy. Each of the features may be autoclavable. Further downstream connections, up to the quick disconnects at the enclosure and not including the pinch valves, are part of the corral and are autoclaved at the same time.

The system or reactor 100 includes a lighting control 170. The lighting control 170 includes a light source 172 to generate light and heat corresponding to an emulated environment. In one example, the lighting control includes a high intensity LED system. The high intensity LED system may include one or more LEDs, for one example 4000K LEDs, to provide a red to blue (R:B) ratio similar to sunlight, such as up to and beyond full sunlight intensity. The light source 172 may vary in intensity to correspond to sunrise, daylight, or sunset (e.g. diurnal). In some examples, the light source 172 is located ˜5-6 in. from bottle to increase homogeneity across front face compared to directly next to the bottle. The light source 172 may generate light distribution down vertical column of the sample bottle with a cover, improving replication of natural (e.g. pond-like) illumination profile.

The reactor 100 may include Gas or pH Controls 180. The Gas or pH controls may provide Air, CO₂, or other gases to the growth chamber 114 to maintain or change a pH of the culture solution or the growth chamber 114. The Gas or pH controller may be associated with a mass flow controller 182 to release the gas, such as air or carbon dioxide, into the growth chamber. The mass flow controller 182 may be operatively connected with a sparger 184 placed within the growth chamber 114. The sparger 184 may release the gas through the culture solution. In some example, the Gas or pH controls may include a glass body pH probe 224 or gas sparger 184. The glass may provide for better optical transmission, such as in comparison to plastic or metallic probes, or a reduction in filming propensity. In some examples, the pH or gas controller administers, by the mass flow controller (MFC) 182, a proportional integral (PI) feedback pH control. During use air strips accumulated oxygen and raises pH. In contrast, CO₂ drops pH. The gas or pH control may disperse either or both of varying concentrations of CO₂ and air.

In some examples, pH control will be achieved using a combination of air and variable CO₂ sparging under proportional control, and measured using the pH probe 224. In one example, the pH probe 224 is Hamilton EasyFerm 160 mm probe. In some examples, the pH or Gas controller 180 circuit can operates in one of two modes: “manual mode,” where air and CO₂ MFC flows are set by the user or a schedule, or “pH control mode” where the proportional controller varies CO₂ flow in the presence of a prescribed air flow to control bottle pH to a set point. The maximum CO₂ flow can be set by the user in software. Other forms of pH control (e.g. windowed) may be programmable in software. In one example, the MFCs 182 for air and CO₂ may be installed in the single stations for manual gas control, and may be tunable using a dial on the face of the instrument.

The reactor or system 100 may include a weigh module or dilutions control 206. The dilutions control 206 may include a weighing module 200 including a mass sensor or load cell 204 to measure a mass of the culture vessel 114. The mass of the culture vessel 114 may be associated with or used to determine a volume of the culture solution. For example, variations in weight may be attributed to evaporation, dilution or addition of a volume of liquid to the culture solution, removal of a volume, or portion, of the culture solution or the like. For example, the load cell may determine an amount of evaporation of the culture solution or discrete volumes, or portions, of the culture solution may be sent to be sampled or to waste. The load cell 200 may be used for determining a volume of liquid needed to compensate for evaporation. Dilution controls may include administration of additional volumes of liquid by a non-contact pump 160. Non-contact pumps 160 may be peristaltic pumps or similar. The changes in weight may similarly be associated with changes in concentrations of the solutions. By determining a load cell volume measurement, the parameters of the culture solution may be determined for long term maintenance of culture bottle media concentrations.

The system 100 can include a temperature controller or system 190. The temperature controller can include an enclosure cooling system 194 and a growth chamber 114 cooling system 192.

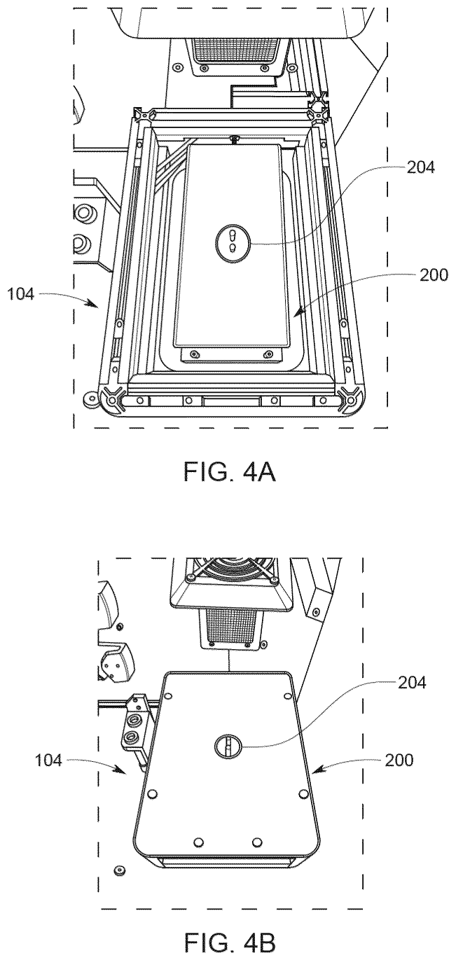

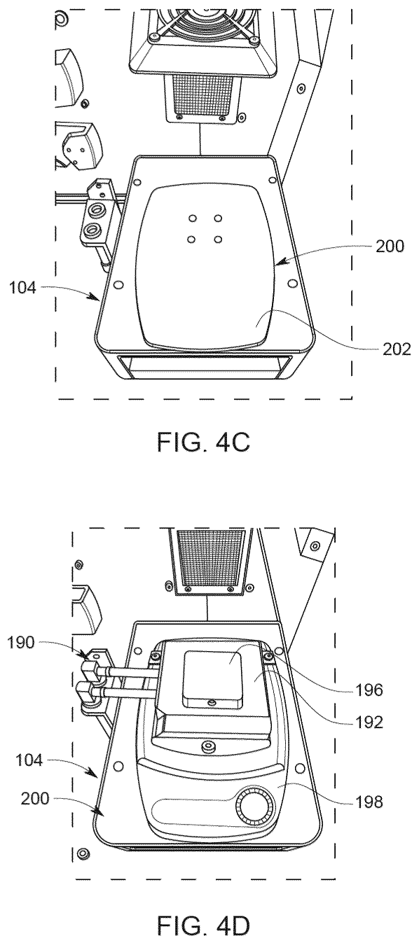

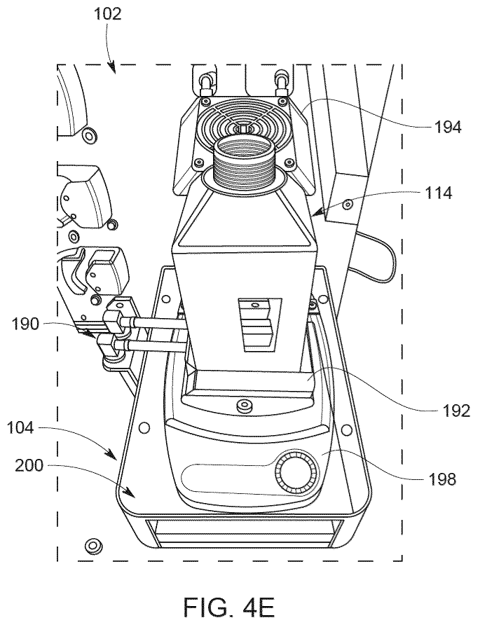

The growth chamber cooling system 192 may include a thermoelectric cooler (TEC), which may provide heat or transfer heat from the culture vessel 114. The temperature control 190 controls temperature across pond-relevant temperatures (e.g. 12-45° C.) at heating/cooling rates observed in outdoor ponds. In some examples, a liquid cooling TEC alleviates need for bulky heatsink and fan on culture bottle platform, and moves heat away from bottle environment 114. The temperature control unit 190 may include aluminum build TEC modules. Aluminum TEC modules 190 may offer greater resilience to mechanical stress compared to ceramic alternatives, which may still be acceptable. In some examples, the temperature control 190 includes one or more silicone thermal pads 196 (exemplified in FIG. 4D) to improve conduction from a thermoelectric cooler (TEC) 192 to the culture bottle 114.

The enclosure cooling 194 can include one or more cooling devices for cooling or maintaining a temperature of the main reactor enclosure 102. For example, the pumps 160, light source 172, mass flow controller 182, the electronics boards 210 described herein, other components may generate heat during use. In some examples, the lighting 172 may be liquid or air cooled for output consistency and to minimize temperature influence on culture solution. The reactor enclosure 102 may include a fan has been added above the LED panel to help regulate panel surface temperature during full sunlight from about 53° C. to about 40-43° C. This also helps decrease ambient temperature within or around the culture vessel 114 environment substantially, casing the load on the temperature controller for the culture vessel 114.

With reference to FIGS. 4A-4E, the growth chamber or culture vessel platform 104 includes one or more components of the weighing module 200, the temperature control 190, or a stir plate 198.

In some examples, the weigh module 200 itself sits underneath or within the platform 104. The weigh module may include a weigh platform 202 defining a growth chamber floor of the platform 104. The mass sensor or load cell 204 may support the weigh platform 202, such as at aweigh point, as exemplified in FIGS. 4A-4C. In some examples, the thermoelectric cooler 192 may be placed on the weigh platform 202. In some examples, the TEC 192 is positioned on a stir plate positioned on top of the bottle platform (FIG. 4D). The stir plate may be a magnetic stirrer or stir plate to assist in mixing or inducing movement in the culture vessel 114 without unsealing or exposing the culture vessel 114. In some examples, a silicon thermal pad, or other heat transfer component, may be positioned between the TEC and a culture vessel 114. By placing the culture vessel on the TEC 192, or weigh platform 202 generally, bottle mounting may be simplified, multiple functions (e.g. mixing, mass measurement, temperature control) and reductions in light reflections may be accomplished without moving or disturbing the culture vessel 114.

The system 100 includes one or more sensors 220 to monitor various environmental parameters and liquid conditions and/or manipulations. For example, the system 100 may include one or more of optical density (OD) sensors 222, the pH probe 224, or temperature sensors 226.

The OD sensor 224 may connected to the cap 116 or placed into the growth chamber 114. In one example, a mini dip transmission probe from Avantes may be employed. The OD sensor 224 may measure optical density on demand without removing sample 132 from the culture bottle 114. In some example, 970 nm wavelength LED output may be coupled into a fiber optic cable and split either 50:50 or 90:10 (depending on whether a typically dilute or dense culture is being measured, respectively). The larger or higher intensity portion of the light (e.g. a majority signal) may be piped or directed into the culture bottle 114 across an air gap transmitter. The air gap transmitter may include two or more collimators that may allow contactless light transmission to the culture bottle platform 104. Fiber optic cables may be rigid or less compliant than, for example plumbing 140 connections, made on the platform 104. An air gap transmitter may reduce or eliminate varying loading effects from fiber optic cables otherwise directly connected to the bottle 114. The collimators may be connected to the cap 118. The majority signal will then pass through to the OD sensor 224, which passes light across a distance, (e.g. 5 mm gap), to a mirror that reflects light back into the receiving fiber, for a total 2× (e.g. 10 mm) path length in the culture. This transmitted light may then passed across another air gap transmitter to the sample photodiode. The minority signal, or less intense or split portion of the light, may be sent back to the reference photodiode, and both signals will be read in a logarithmic amplifier setup to produce a voltage proportional to optical density. In some examples, the optical density sensor 224 may be or include a Hastelloy probe tip design to maintain rigidity and robustness while maintaining corrosion resistant to high salinity media similar to sea water.

The sensors 220 may include a temperature sensor 226. The temperature sensor 226 may be a thermistor. The temperature sensor 226 may have a shaft style or a bead style assembly. In some examples, a smaller or bead thermistor may be selected, as temperature measurements of smaller volume cultures (about 200 mL or less) may become more susceptible to temperature conduction effects along the shaft in thermal contact with the side of the bottle surface 114 not below the volume surface.

The system and devices may include one or more components that may aid in containing spills (not shown). In some embodiments, the disclosed culture vessel may be seated or contained on or within a containment bin, the containment bin may be fitted with various structures to hold and secure the vessel 114 and/or main enclosure 102 and related components, and may include a bulkhead quick disconnect port to direct any spills to a tank, for example a waste tank.

The system 100 may include one or more electronics boards 210. The electronics boards 210 may be a stackable printed circuit board (PCB) system. The electronics boards 210 may be modular for upgrades, replacements, or a reduced storage volume in the enclosure 102.

The electronics boards 210 may include one or more processing elements 212. The processing elements 212 may execute or generate one or more operations such as scheduling, analysis of captured data, or other operations. In some examples, the processing elements 212 may be Arduino's or Raspberry Pi. In some examples, the electronics boards 210 or processing elements may be programmed, such as by using LabVIEW, directly. Software and schedules can be run heedlessly (e.g. without a user interface or inputs).

The electronics boards 210 can include a communications module 214 or interface. The communications module 214 may wirelessly, or by wire connection, access a network 108 to communicate, transfer, or receive information from one or more devices. For example, communication with a server 110 may identify or transfer relevant information. A remote device 112 may generate commands, schedules, or otherwise be in operative communication with the main enclosure 120. In some examples, the system may include one or more Raspberry Pi's that can also communicate wirelessly, allowing more freedom in lab setup. For example, a Raspberry Pi may transmit UART in addition to the SPI protocol.

The electronics board 210 can include a weigh module board or controller 190. The weigh module board 190 may be a level shifter for the UART (RS232) signals used to communicate with the WKC6002C. The mLVDS ICs accept and transmit 3.3V CMOS voltages and must be converted to ±5-15V. This may done using the MAX3232 IC.

The electronics board 210 may include a light controller or LED control board 170. The LED control board 170 in some examples may be TPS92641 LED driver IC and a LTC6992 signal generation IC. In other examples, the board 170 employs a MAX16833 LED driver IC and more common signal generation implementation using op amps.

The electronics board 210 can include temperature controller or temperature control board 190. In one example, the board may be a LTC1923 TEC driver IC. In other examples, the temperature control board 190 may be DRV595, or other more readily available boards as a more readily available IC.

In some examples, the electronics boards 210 may be separate or portions of the same board. In one example a board 310 has sections for the LED driver, TEC driver, and absorbance measurement circuit. The LED driver powers the light source 172. In one example, the LED drive controls the 970 nm wavelength LED to approximately 60% of full current, as greater intensity may results in diminishing returns. The TEC driver may regulate the temperature of the light source 172 to maintain various subcomponents at similar temperatures, which may be slightly below ambient. This helps match outputs of the photodiodes for consistent measurements across varying ambient conditions and reduces dark current.

The reactor housing 102 may include one or more power sources 230. The power sources 230 may be separated to provide electronics power 232, such as to the electronics board 210, or enclosure power 234 other components of the system 100. The power source may have varying voltages, wattages, or amperes. In one examples, a 4-slot chassis houses a 12V 20 A card, a 48V 6 A card, and two 24V 9 A cards. A separate 12V 5 A supply powers fans and liquid cooling pumps. In one example, power is provided by an AC-DC power supply, such as Excelsys Xgen from Advanced Energy. In some examples, the system 100 is positioned in proximity to one or more other systems 100. In such an example, universal power system (UPS) may be integrated into a rack that houses a plurality of single station growth chambers 102 to reduce the risk of power loss from external energy sources

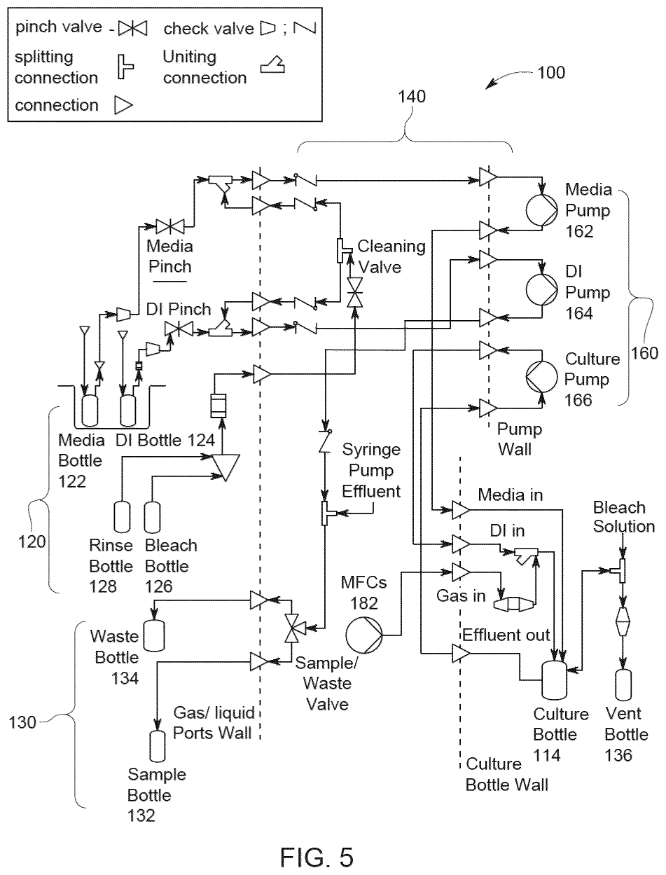

With reference to FIG. 5 a detailed example of the system 100, including the plumbing is depicted 140. Media 122 and water, for example DI water (“DI”), 124 may pulled from sterile vessels (upper left) by peristaltic pumps 160 (depicted upper right) and fed to the culture vessel 114 (bottom right). The pumps 160 can include a media pump 162 for dispensing media 122. The pumps 160 can include DI pumps 164 for dispensing dilution 124. The bleach solution 126 and rinse bottles 128, may be connect to the enclosure 102 or plumbing by one or more connections, such as the SeriesLock quick disconnects.

The plumbing 140 can include non-contact pinch valves to selectively limit flow of liquids. The non-contact pinch valves may be positioned prior to a uniting connection or junction (e.g. the Y) along each or either fluid's path. In normal operation, the pinch valves may be disengaged (allowing media/DI flow) and the internal cleaning valve may be engaged (restricting flow).

When performing sterilization, external pinch valves and cleaning valves may be activated, for example engaged, and disengaged, respectively. The cleaning valve may be placed between the plumbing 140 of the media 122 or DI 124 and the cleaner 126 and rinse 128. Opening or disengaging the cleaning valve allows pumps to draw from either bleach solution 126 or rinse DI 128. The plumbing between the bleach solution 126 or rinse DI 128 can filtered by an externally mounted 0.2/0.1 μm filter. After a bleach treatment, DI rinse 128 is drawn through the same fluid paths or plumbing 140. Bleach 128 can potentially remain sequestered in the tubing after the external pinch valves prior to the junction with the media or DI plumbing 140. Accordingly, the rinse procedure can switch between rinsing with DI from the rinse bottle 128 and fluids from the media 122 or DI 124.

Sample and waste plumbing may function similarly. A culture effluent pump 166, such as a peristaltic pump, may selectively withdraw culture solution for waste or sampling. For example directing the withdrawn culture solution (or “effluent”) to a waste bottle 134 or sample bottle 132. Effluent may be drawn by the culture pump 166 and routed either to a sample bottle 134 or to waste 132. A sample or waste valve may selectively direct effluent from the culture bottle 114 to the waste bottle 134 or sample bottle 134. In some examples, the system 100 can include a manual sterilization or rinse port to clean sample lines.

The culture bottle top includes connections for inlet lines and outlet lines of the plumbing 140. The culture bottle cap 116 includes new quick disconnects at the connections, such as the SeriesLock disconnects. A sparger 184 may be positioned in the culture vessel 114. A mass flow controller 182 may selectively release gas, such as air or carbon dioxide, into the vessel to change or maintain a pH of the solution. In some examples, gas is passed through a 0.2 μm syringe filter and coupled into the DI inlet line to aid in dissolving salt buildup in the sparger 184 that may be submerged in the culture liquid.

During operation, the culture vessel 114 can accumulate gases or internal (e.g. headspace) pressure. The headspace pressure may be equilibrated by means of a vent bottle 136 and a filtered atmospheric inlet. Gas exiting the culture bottle 114 may be bubbled through bleach or the cleaning solution in the vent bottle 136 as a barrier from contamination, such as to other systems 100 or a lab environment. In one example, the vent bottle includes 136˜20 mL of bleach or the cleaning solution. The vent bottle 136 outlet path is checked to prevent back flow of the vent bottle 136 solution, or released gas. In some examples, negative headspace pressure equilibration may be facilitated by passive filtered intake from the connections 118 defined by the cap 116.

The operation of the plumbing 140, such as the pumps 160, can be determined by the electronics board 210, such as the processing element 212. In some examples, a hit and hold circuit is added to a valving driver circuit, as may be executed or defined by one or more of the electronics boards 210, to prolong lifetime and performance. A hit and hold circuit may drive the valve initially with 100% power for an initial duration, in one example ˜50 ms (hit) to move the valve. The hit may be followed by a drop in pressure, such as to 50% drive power, to maintain that state (hold). This reduces the heat generated by the valve coil, mitigating potential thermal degradation of the Teflon body.

At operation, the culture vessel 114 maybe operatively connected to the main enclosure 102 by plumbing 140. The culture vessel 114 may be filled with a culture solution and have a size or volume corresponding to a pond voxel. In some examples, the culture vessel 114 may be filled with an initial solution before or after coupled to the enclosure 120 or plumbing 140. In some examples, the culture vessel 114 may be positioned on the platform 104 and operatively connected to one or more of the weigh module 200, temperature control module 190, 192, light source 172, or the gas or pH control 180. For example, the components may be connected to the cap 118.

The electronics 210, such as the processing element 212, may execute or determine a schedule to control one or more of a pH, temperature, liquid volume, dilution, or light intensity of the culture vessel 114. The schedule or program may define an intended light intensity or temperature of the culture vessel 114 at various time period. During operation, the schedule or operation may be maintained to define culture vessel 114 environment similar to outdoor ponds. In such an example, the light intensity, temperature, or other parameters may correspond to a geographical location. For example, the parameters may be varied or maintained at values, or between ranges of values, to simulate varying environmental conditions. In one example, the parameters may be adjusted to correspond to day and night cycles. The sensors 220 may determine or identify the parameter (e.g. light intensity, a temperature, mass) of the culture solution and automatically adjust to culture conditions.

In some examples, the enclosure 102 may include one or more input devices, such as dials, keypads, or the like. In such an example, the input devices may enable an operator to operatively select or modify one or more parameters of the culture vessel 114. For example, the user may change or set a temperature, pH level, gas flow rate, or the like.

In some examples, a temperature of the culture vessel 114 may be controlled by the temperature controller 190. The temperature sensor 226 may be used to monitor the temperature of the vessel 114. To increase a temperature, such as to a desired temperature or temperature set point, the power provided to the TEC 192 may be reduced or light intensity from the light source 172 may be increased. To reduce a temperature, the power provided to the TEC 192 may be increased or light intensity from the light source 172 may be decreased. For example, the schedule or program may cause the temperature controller 190 to cool the culture vessel 114 by a thermoelectric cooler 192 to adjust the temperature to a simulated temperature corresponding to the light intensity and the geographical location. In some examples, dilutions, such as from the media 122 or DI 124 may be added to the vessel 114, such as at timed or manually determined events, to increase, decrease, or maintain vessel 114 temperatures.

The pH levels of the culture vessel 114 or culture solution may be adjusted or maintained, such as by the gas or pH controller 180. For example, a pH level, pH may be measured by the pH probe 224 to determine if the pH value is above or below a desired value. In such an example, the gas or pH control 180 may release gas through the culture solution to increase or decrease the pH. For example, the sparger 184 may release gases through the culture vessel 114. Sparging one of air or carbon dioxide within the culture solution to increase or decrease, respectively, a pH of the culture solution. Depending on the culture solution, or media and other inputs 120 added to the culture solution, in some examples the gas and pH controller 180 may provide only or both air or carbon dioxide by the sparger 184. In some examples, pH may be maintained at predetermined values or ranges of values. In such an example, gas may be sparged after pH levels exceed or fall below an intended value. In other examples, gas may be sparged at predetermined intervals or in anticipation of the culture solutions having pH values outside of the range.

During operation, a mass of the culture vessel 114, and including the culture solution, may be determined by the weigh module 200. The mass may be associated with a volume of the culture solution. In some examples, the identified or determined mass or volume may indicate a need to release or increase a volume of the culture solution. In such an example, an inlet solution (e.g. media, dilution) may be added to the culture vessel 114, or sample 132 or waste solution 134 may be released by the plumbing 140 to change the volume of the culture solution. In some examples, solution may be added and then removed from the vessel 114, or removed and then added to the vessel 114.

During operation, characteristics of the culture solution may also be determined. For example, the optical density sensor 222 may be used to determine the optical density of the culture solution. The optical density may be indicative of concentrations of solutes, shapes or sizes of suspended particles, biomass, or the like. Changes in optical density may be used to determine growth rates or other changes of culture vessel 114 conditions. To measure the optical density, the optical density sensor 222 may emit light into the solution. The light may be separated into two or more emissions, such as a control and one into the solution, and reflected back to the sensor 222. The intensity, scattering, absorption, or wavelength of the light of each of the emissions may be measured and compared to determine characteristics of the culture solution. In some examples, the OD sensor 222 may be placed directly into the solution.

### EXAMPLES

In one example, an embodiment of the disclosed system and device, in this case a single station Bioreactor, was constructed and tested. The single station incorporates various components, for example a controller, which may be a Raspberry Pi based controller. The first units controlled light and temperature successfully. Additional capabilities (pH control, OD measurement, and membrane-introduction mass spectrometry (MIMS) autosampling) were tested.

The reactor included a sample vessel or culture vessel for placing the solution for testing or a culture vessel for observing the culture solution over time. The reactor included or positioned components at a bottom or top of the culture bottle to case bottle mounting and reduce light reflections. The culture bottle, in this embodiment, was a modified low cost off the shelf glass sample vessel with flat sides that aided in emulating a pond surface “voxel” or virtual environment. The culture vessel includes a cap with a modular design. The modular cap design provides adaptability to connect with a multitude of analysis probes or other connections.

The reactor included a liquid storage system. The liquid storage system included input solutions such as media, dilution solutions, cleaning solutions, or rinsing solutions.

Power was provided to the reactor by an Excelsys Xgen AC-DC power supply from Advanced Energy. A 4-slot chassis houses a 12V 20 A card, a 48V 6 A card, and two 24V 9 A cards. A separate 12V 5 A supply powers fans and liquid cooling pumps.

A universal power system (UPS) was integrated into a rack that houses a plurality of single station growth chambers to reduce the risk of power loss from external energy sources. The rack provided space for each growth chamber's liquids corral, sample and waste bottles, and sterilization bottles, as well as gas manifolds for distribution to each station. Each reactor will sit in a containment bin fitted with a bulkhead quick disconnect port to direct any spills to a common tank.

In the present example, the reactor includes a Lighting Control. The lighting control included a High intensity LED system. The lighting control was liquid and air cooled for output consistency and to minimize temperature influence on culture. The High intensity LED system included 4000K LEDs to provide a red to blue (R:B) ratio similar to sunlight, such as up to and beyond full sunlight intensity. The LED system was located ˜5-6 in. from bottle to increase homogeneity across front face compared to directly next to the bottle.

During testing optical measurements with Walz spherical light probe showed even light distribution down vertical column of the sample bottle with a cover, improving replication of pond-like illumination profile. OD (Optical Density) measurements were taken. OD measurements were captured using a Hastelloy probe tip design to maintain rigidity and robustness while maintaining corrosion resistant, such for use with high salinity media similar to sea water.

In one example, the reactor included a temperature control. The temperature control maintains and controls temperature of the culture solution across pond-relevant temperatures (e.g. 12-45 C) at heating/cooling rates observed in outdoor ponds. The reactor included silicone thermal pad to improve conduction from a thermoelectric cooler (TEC) to the culture bottle. A liquid cooling TEC alleviates need for bulky heatsink and fan on culture bottle platform, and moves heat away from bottle environment.

The reactor includes Gas or pH Controls. The Gas or PH controls may provide Air or CO₂ administration by mass flow controller (MFC) for a proportional integral (PI) feedback pH control. During use Air strips accumulated oxygen and raises pH, while CO₂ drops pH. The Gas or PH controls may include a glass body pH probe and gas sparger for better optical transmission than plastic probes and reduction in filming propensity.

The reactor included a Dilutions Control. Dilution controls may include administration of additional solutions by a non-contact peristaltic pump. Portions of the solution may be sent to sample or waste containers. The reactor may include nearly or mostly metal-free paths. The metal-free paths reduce or eliminate impact of corrosion on parts from saline media, and maintains liquid component integrity for longer periods. The various paths may include or liquids contact platinum-cured silicone, PVDF, PTFE, glass, chrome-plated stainless steel 316L, titanium, polypropylene, bioprene, polyethersulfone, PEEK, silicone rubber, or viton. The paths may include embedded tubing to reduce static charges within metal free path. The paths include filtered inlets, outlets, or other vents.

The culture vessel is magnetically stirred. A stir plate is positioned below or in contact with the culture vessel to stir the culture solution. The dilutions control may include a load cell to measure a mass of the bottle. Load cell volume measurement allows long term maintenance of culture bottle media concentrations. The load cell may determine an amount of evaporation of the solution. The load cell may be used for determining compensation for evaporation.

The reactor includes electrical systems to generate or execute commands or to receive or store information from the components. The electrical systems are designed for modularity. The electronics are in a stackable printed circuit board (PCB) system. The electrical components include one or more processing elements for headless operation. National Instruments had officially began supporting Raspberry Pi's, which offer numerous advantages over the previous Arduino base in the first iteration of the main board. The Raspberry Pi can be programmed using Lab VIEW directly, and software and schedules can be run headlessly. Raspberry Pi's can also communicate wirelessly, allowing more freedom in lab setup. The main board has been updated to connect to a Raspberry Pi and transmit UART in addition to the current SPI protocol.

### Lighting and Temperature Control

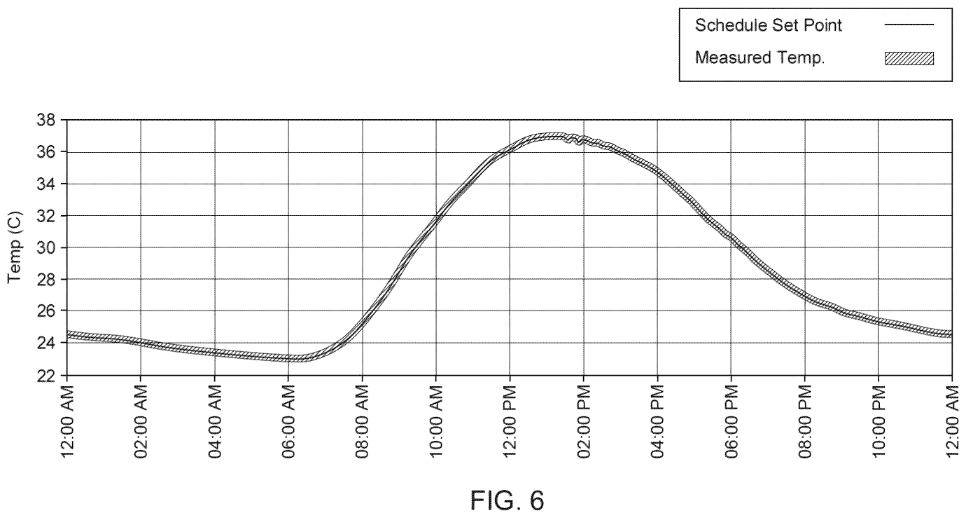

With reference to FIG. 6, low-profile components have been successfully tested on both the LED and TEC boards. Elements produce slightly more heat during operation as the new components exhibit increased resistance, though were tested without cooling. Stable low light illumination has been demonstrated as low as 11 μmole PAR with no flicker and no compromise to maximum intensity (˜3250-3500 μmole PAR). The shaft-style bottle thermistor has been replaced with a smaller bead thermistor, as temperature measurements of smaller volume cultures (˜200 mL) become more susceptible to temperature conduction effects along the shaft in thermal contact with the side of the bottle surface not below the volume surface. Bottle temperature measurements now agree within +/−0.2-3° C. of liquid temperature depending on the temperature ramping, and typically within 0.1° C. at steady state. The temperature control unit features the new aluminum build TEC modules that offer greater resilience to mechanical stress compared to their ceramic alternatives.

The reactor enclosure shown includes a fan added above the LED panel to help regulate panel surface temperature during full sunlight from ˜53° C. to ˜40-43° C. This also helped decrease ambient temperature within the bottle environment substantially, casing the load on the temperature controller.

### PH/Gas Control

Hardware pH control will be achieved using a combination of air and variable CO₂ sparging under proportional control, and measured using a Hamilton EasyFerm 160 mm probe. The circuit operates in one of two modes: “manual mode,” where air and CO₂ MFC flows are set by the user or a schedule, or “pH control mode” where the proportional controller varies CO₂ flow in the presence of a prescribed air flow to control bottle pH to a set point. The maximum CO₂ flow can be set by the user in software. Other forms of pH control (e.g. windowed) will be programmable in software. MFCs for air and CO₂ have been installed in the single stations for manual gas control, tunable using a dial on the face of the instrument.

### Weigh Module and Board

The weigh module board is a level shifter for the UART (RS232) signals used to communicate with the WKC6002C. The mLVDS ICs accept and transmit 3.3V CMOS voltages and must be converted to ±5-15V. This is done using the MAX3232 IC. The weigh module itself sits underneath the bottle stage floor and supports the bottle platform at the weigh point. The TEC assembly and stir plate mount on top of the bottle platform, as discussed with reference to FIG. 4A-4E.

### Motor/Valving Board

The board now features TMC2660C motor drivers, with step and direction control. As well, a hit and hold circuit has been added to the valving driver circuit to prolong lifetime and performance. This serves to drive valves initially with 100% power for ˜50 ms (hit) to move the valve, followed by a drop to 50% drive power to maintain that state (hold). This reduces the heat generated by the valve coil, mitigating potential thermal degradation of the Teflon body.

### Plumbing

A basic overview of the plumbing is shown in FIGS. 2 and 5. Media and DI are pulled from sterile vessels by peristaltic pumps and fed to the culture bottle. Effluent is drawn by the culture pump and routed either to a sample bottle or to waste. Some culture bottle top quick disconnects were a 4-port stainless steel build, which satisfied most compatibility and function requirements, but showed gradual degradation on the sealing surfaces and led to connection reliability problems. External liquid connections are now corrosion resistant, in one example made by SeriesLock Quick Disconnect Fittings from Eldon James. The liquid connections are autoclavable, polysulfone quick disconnect-to-barb fittings which feature a spring-free flow path, allowing for a completely metal-free path for all liquids in the instrument.

The liquids corral or plumbing includes media and DI vessels with 0.2/0.1 μm Polycap filtered inlets and outlets, 0.2 μm filtered vent, and a stainless steel caddy. Filters mount to the inner rungs of the caddy, and bottles fit between them. This cases setup by making the entire corral assembly autoclavable in one run. Further downstream connections, up to the quick disconnects at the enclosure and not including the pinch valves, are part of the corral and are autoclaved at the same time.

The liquids corral, along with the bleach solution and rinse bottles, connect to the enclosure with the quick disconnects. Non-contact pinch valves are positioned prior to junctions of flow paths along each fluid's path, such as after the liquids corral. In normal operation, the pinch valves are disengaged (allowing media/DI flow) and the internal cleaning valve is engaged (restricting flow). When performing sterilization, external pinch valves are engaged and the cleaning valve is disengaged, allowing pumps to draw from either bleach solution or rinse DI, filtered by an externally mounted 0.2/0.1 μm filter. After a bleach treatment, DI rinse is drawn through the same fluid paths. Bleach can potentially remain sequestered in the tubing after the external pinch valves prior to the junctions, so the rinse procedure will switch between rinsing with DI from the rinse bottle and fluids from the liquids corral.

Sample and waste plumbing function similarly. An effluent pump, such as a peristaltic pump, selectively withdraws culture solution for waste or sampling. A manual sterilization/rinse port allows cleaning of newly attached sample lines.

The culture bottle top includes connections for inlet lines and outlet lines. The culture bottle top includes new quick disconnects at the connections, such as the SeriesLock disconnects. Gas is passed through a 0.2 μm syringe filter and coupled into the DI inlet line to aid in dissolving salt buildup in the sparger/tube that is submerged in the culture liquid.

Bottle headspace pressure is equilibrated by means of the vent bottle and filtered atmospheric inlet. Gas exiting the culture bottle is bubbled through ˜20 mL bleach in the vent bottle as a barrier from contamination. That outlet path is checked to prevent back flow. Negative headspace pressure equilibration is facilitated by passive filtered intake from the upper right fitting assembly.

### Example—Liquids Handling

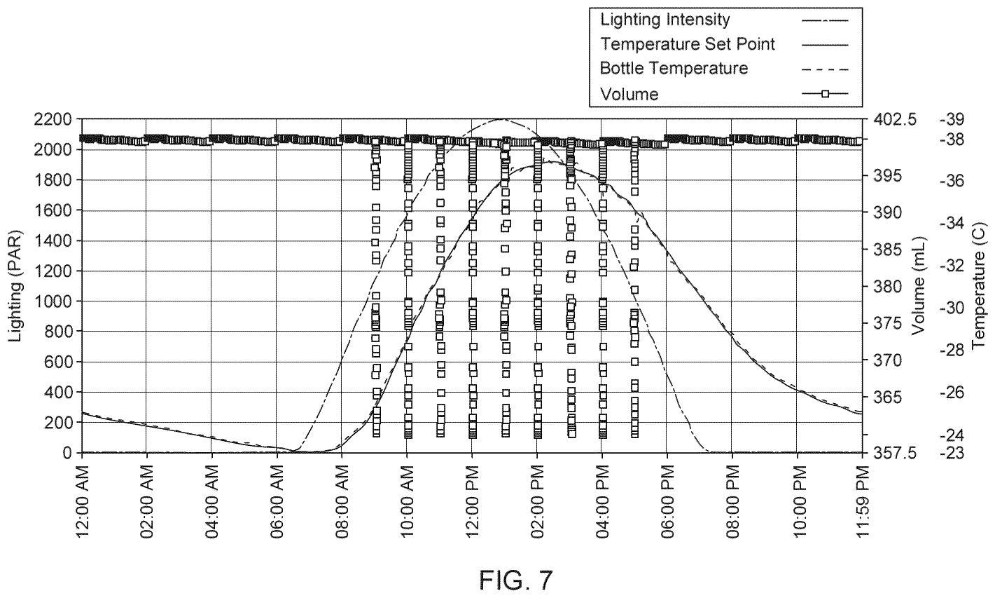

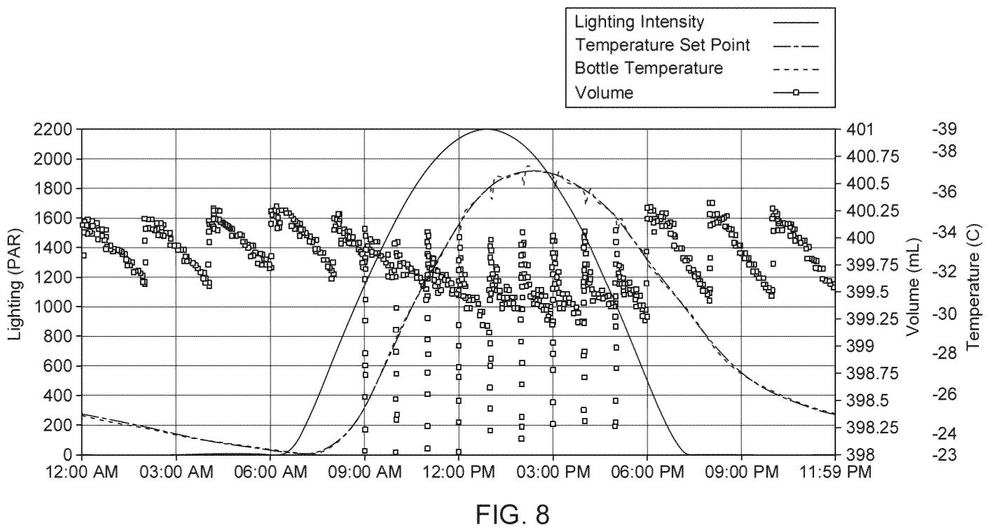

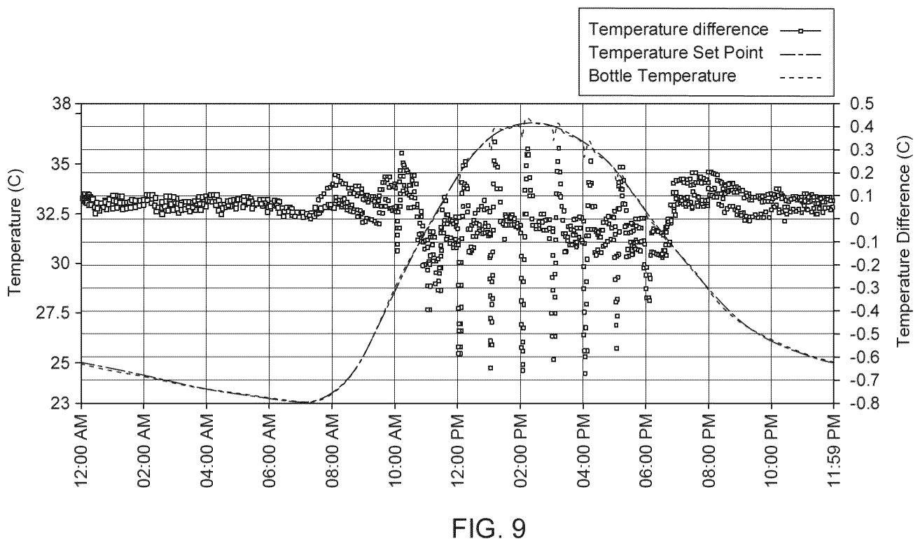

Automated dilutions in the single station growth chamber have been tested, as shown in FIGS. 7-8. The pump electronics controlling the 3 peristaltic pumps allow for ˜0.25-250 mL/min flow rates into and out of the culture bottle with a set point accuracy of +/−˜0.25 mL. Plots demonstrating liquids handling performance are discussed herein.

With reference to FIG. 7, a single station log is shown. FIG. 7 depicts diurnal light and temperature with hourly daytime 40 mL dilutions and dilution (DI) replenishments.

FIG. 8 depicts nominal volume set point variances with respect to temperature swings. The temperature swings are in response to liquid removal and additions from the culture vessel. In this example, culture vessel fill set points of 400 mL are achieved with a typical 0.25 mL overshoot. Applicant notes the weigh platform sensitivity to temperature control impacts volume measurement by approximately +/−0.4 mL in this example.

The results of FIGS. 7 and 8 indicate the system adheres to intended parameters and accurate volumes were maintained during testing.

### Example—Temperature Control

With reference to FIGS. 9-11. Temperature performance of the single station growth chamber 100 are shown below. Dilutions induced small changes in culture bottle temperature that resolved in ˜20 minutes.

FIG. 9 depicts 40 mL dilutions. The 40 mL dilutions induced +0.3/−0.7° C. swings in measured bottle temperature during diurnal temperature control and about 23-25° C. ambient temperature. Temperature control outside of dilutions was consistently at +/−0.1-0.2° C. of set point.

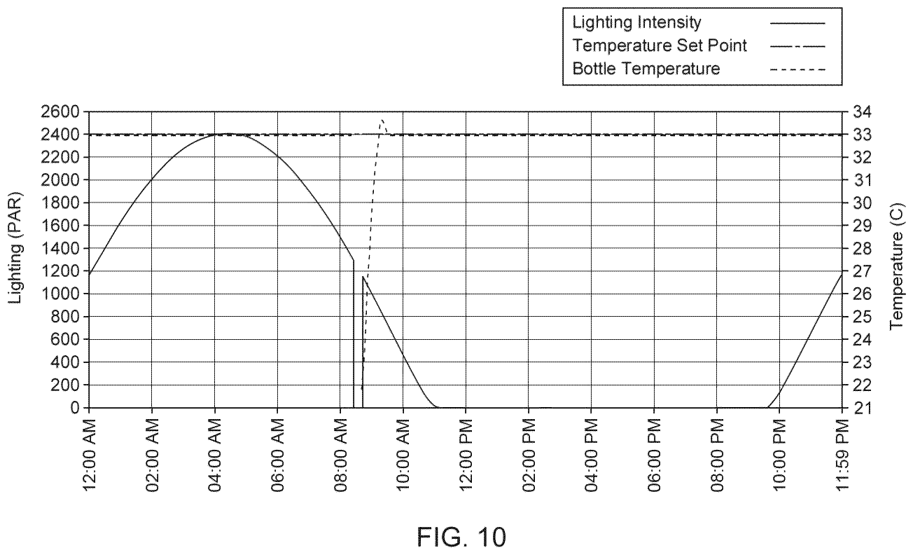

FIG. 10 depicts constant 33° C. temperature control with a single manual dilution. In response to a dilution the temperature control initially overshoots the target temperature, in one example by ˜0.5° C. initially before settling.

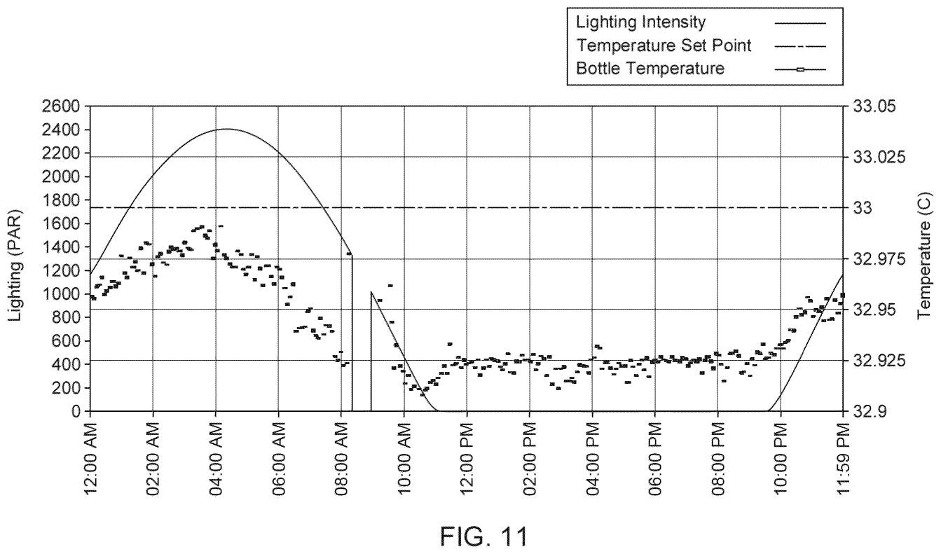

FIG. 11 depicts constant temperature control variance with respect to a diurnal light change. FIG. 11 also depicts a single manual dilution. Temperature varies by less than 0.1° C. over the 24 hour period.

### Example—Emulating a Geographic Location

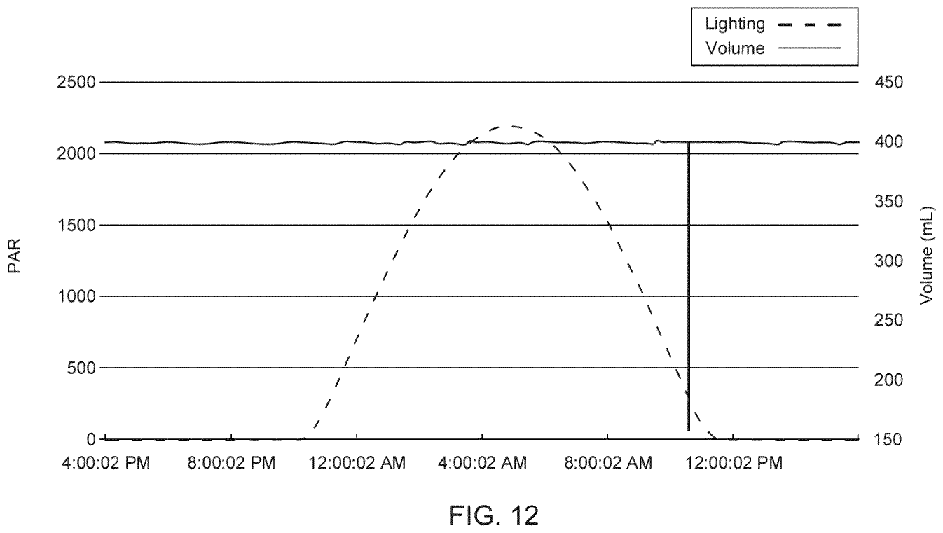

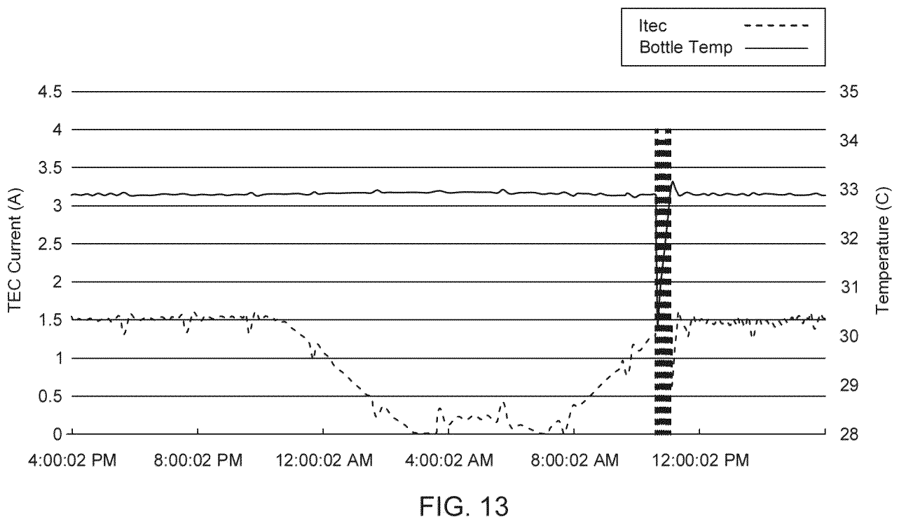

With reference to FIGS. 12-13, a log of the single station is depicted. The single station was running a June 15th Mesa, AZ light schedule. The temperature control was set to 33° C. constant temperature. And a daily 60% dilution was made before sunset with respect to the light schedule. Evaporative losses were replenished every 2 hours. FIG. 12 is a single station bioreactor lighting and bottle volume log depicting volume of the culture bottle volume and the intensity of the light source. FIG. 13 is a single station bioreactor bottle temperature and TEC current log depicting the temperatures of the thermoelectric cooling device and the culture vessel against an electrical current value (e.g. power consumption) of the thermoelectric cooling device. During testing, the temperature remains within 32.9-33° C. during the diurnal light curve, with exception of the dilution period.

### Example—PH Control

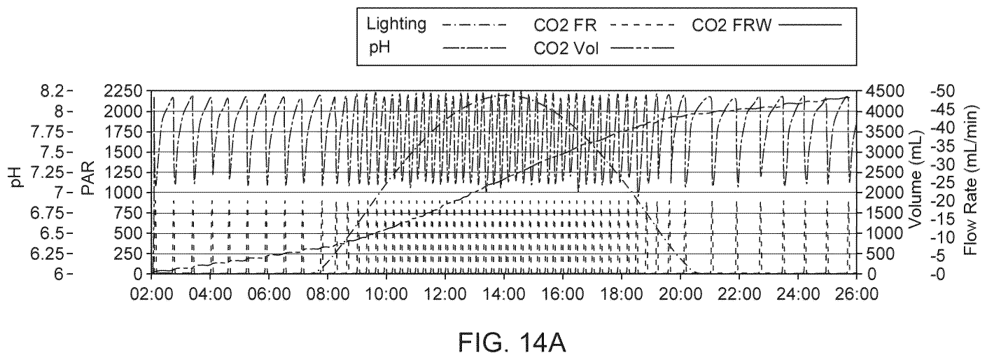

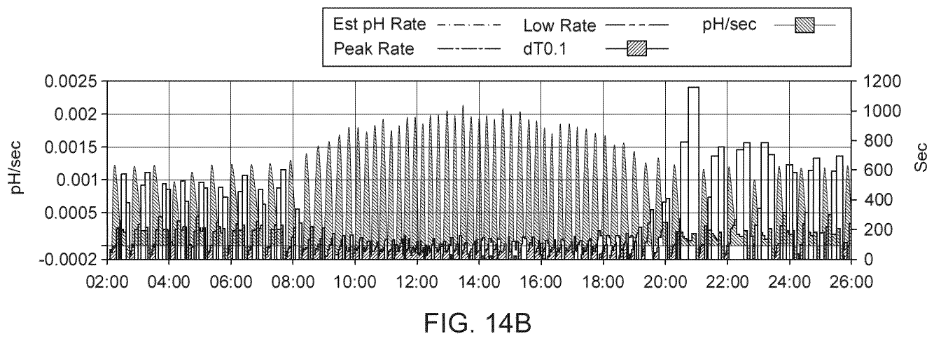

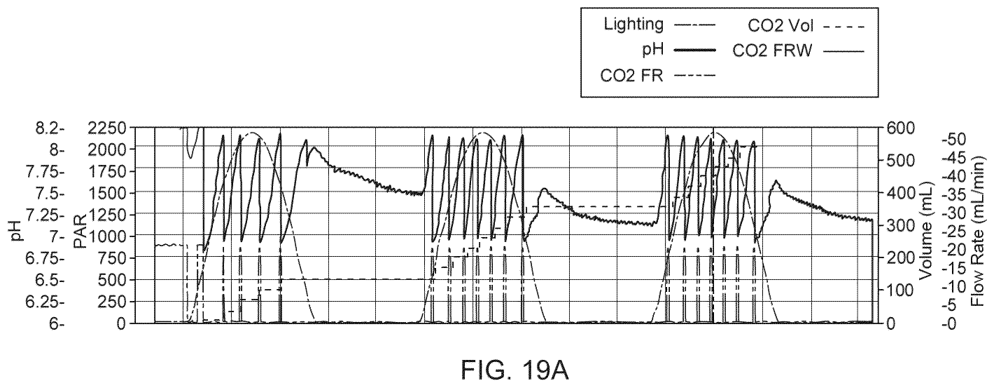

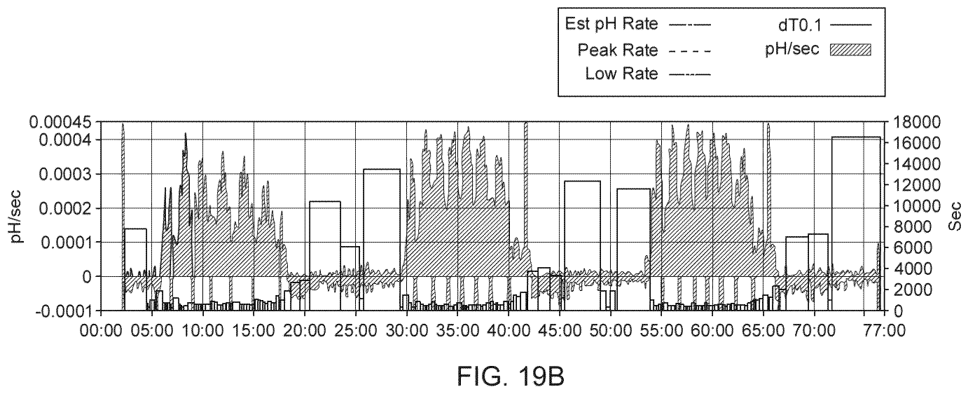

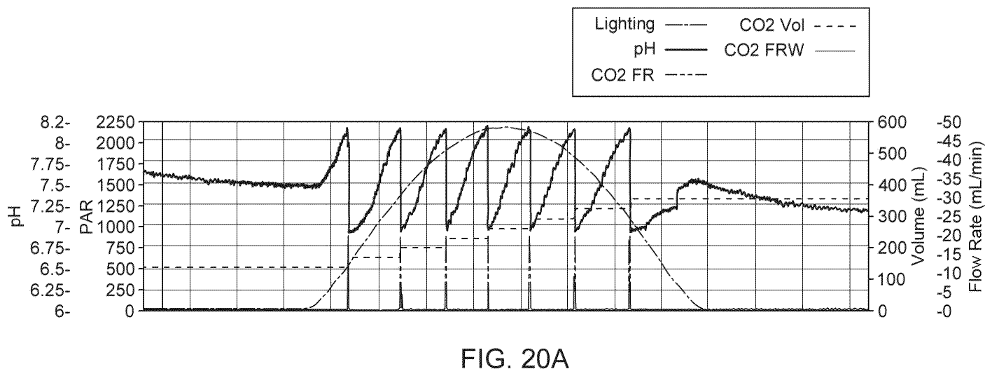

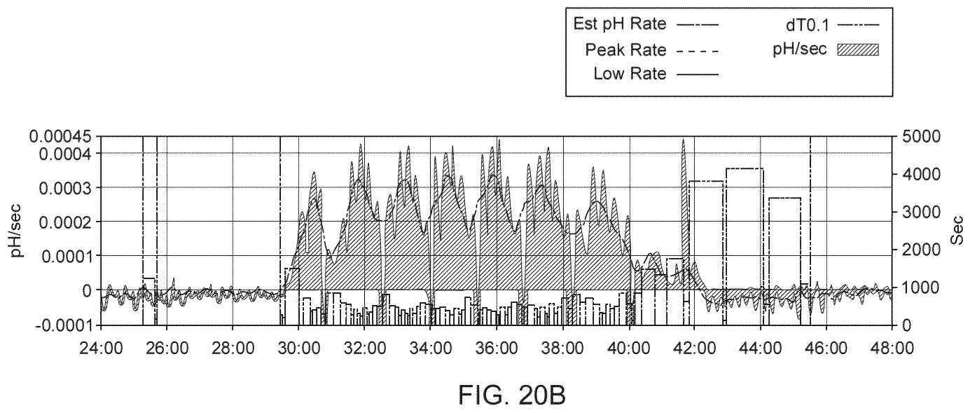

pH boards have been tested with picochlorum in a variety of pH control schemes, as shown in FIGS. 14A-20B. FIGS. 14A and 14B depict picochlorum grown under a June 15th Mesa, AZ day at 33C with 60% daily dilutions after sunset. The culture vessel and culture solution underwent a bounded pH control scheme between pH 7 and 8.2 with 400 mL/min air background sparging.

In FIG. 14A, pH is shown to rise rapidly due to the additional air sparging, and when upon hitting pH 8.2, the pH control circuitry is engaged to drive the pH down to 7. Carbon Dioxide (CO₂) flow rate and the integrated sparged CO₂ volume is also shown. A frequency of pH swings increases towards the peak of the day, as shown in FIG. 14B, where each bar represents the time it takes for the pH to change 0.1 units.

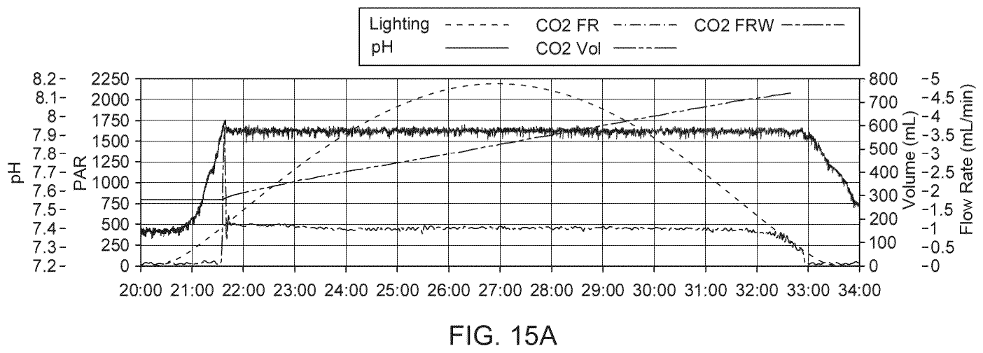

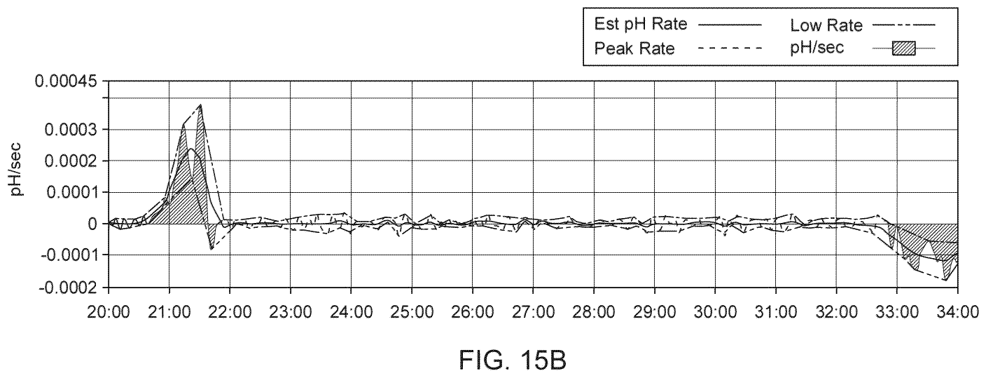

In FIG. 15A-17B, picochlorum is grown under the same light, temperature, and dilution schedules as above, but with a pH control set at pH 7.9 and without an air background. pH adjustments were made with pure CO₂ sparging.

Small nonlinearities, which may be an artifact of the pH probe are visible at certain voltages. These small nonlinearities lead to small bumps in pH readings in FIG. 15A, and larger bumps in the pH rate calculation in FIG. 15B. Compensation is attempted in software in the estimated pH rate trace to mitigate some of the offset.

The estimated pH rate is an average of the peak and trough pH rates (low rate) in FIG. 15B. The windowed average of the CO₂ flow rate is represented in FIG. 15A (Est pH Rate). There is also a degree of noise present on the pH trace, which has been addressed recently in hardware (though not reflected in the presented data). Based on Hamilton's Arc Air software, during control the pH holds at 7.90 +/−0.01.

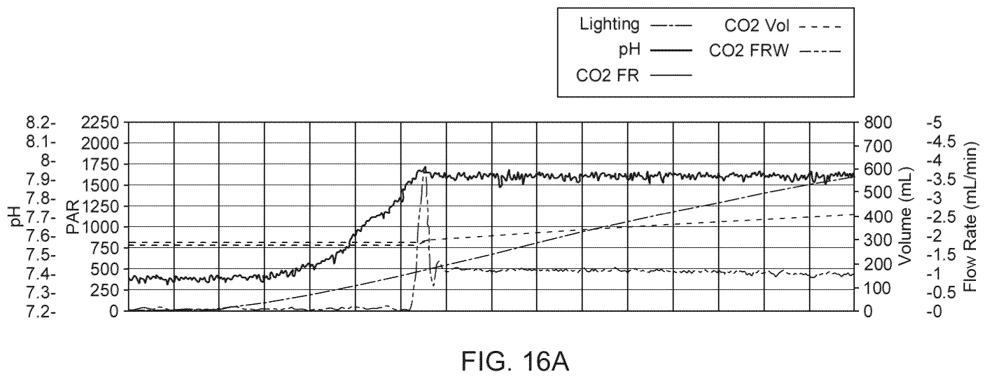

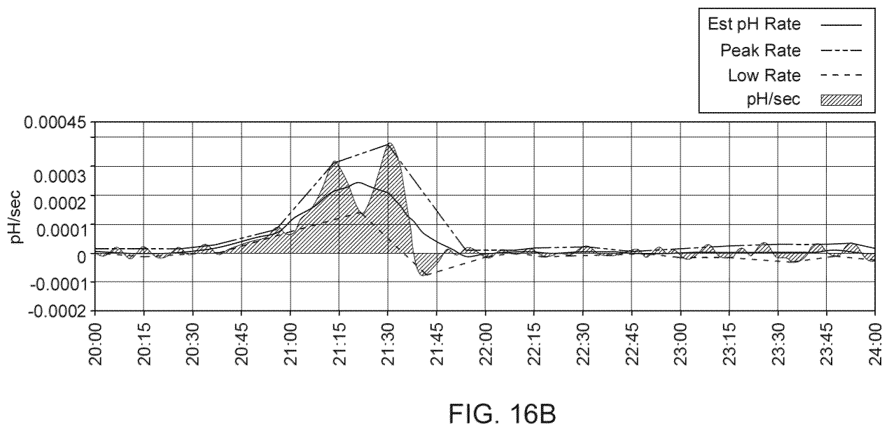

FIGS. 16A and 16B show similar testing conditions. FIG. 16A and FIG. 16B depict a pH control set at 7.9 and without an air background. pH adjustments were made with pure CO₂ sparging. In FIGS. 16A and 16B, the light intensity was comparable to a morning light intensity.

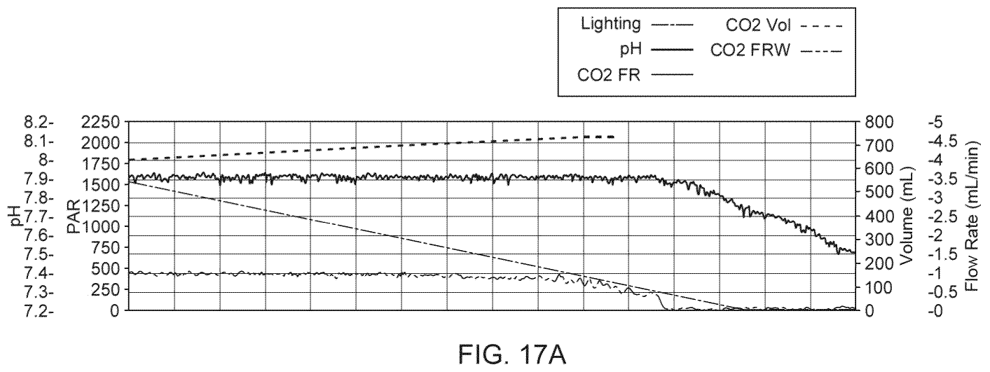

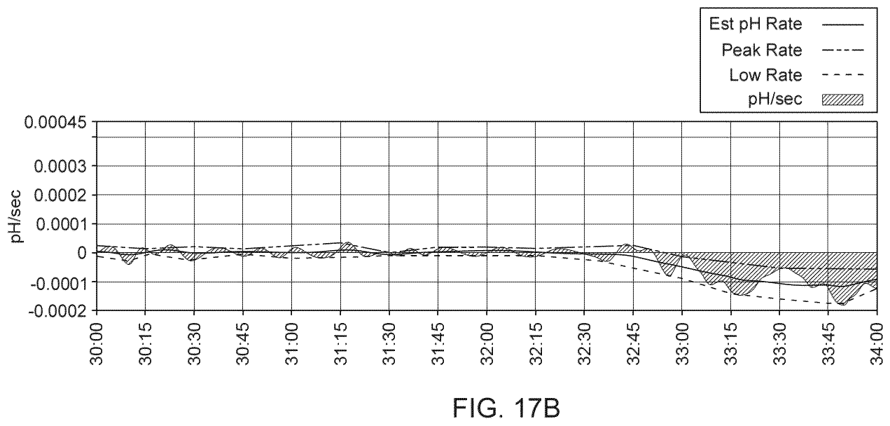

FIGS. 17A and 17B show similar testing conditions. FIG. 17A and FIG. 17B depict a pH control set at 7.9 and without an air background. pH adjustments were made with pure CO₂ sparging. In FIGS. 17A and 17B, the light intensity was comparable to an evening light intensity.

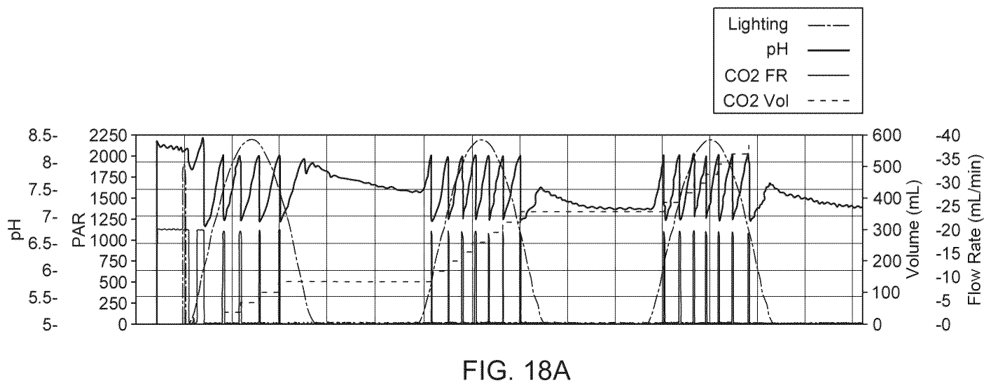

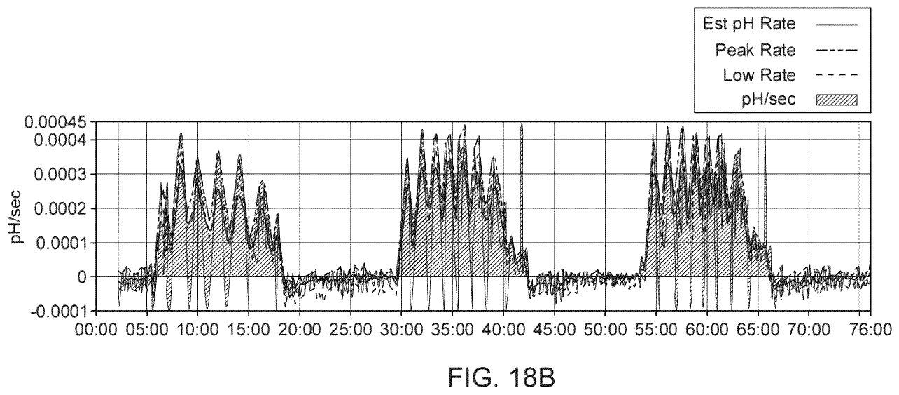

In FIGS. 18A-20B, picochlorum is again grown with the same lighting, temperature, and dilution schedules, respectively with reference to FIGS. 15A-17B. However, in FIGS. 18A-20B the picochlorum is grown with bounded pH control between 7-8.2 and no air background. The pH adjustments were made with pure CO₂ upon surpassing pH 8.2. Windowed CO₂ flow rates are not shown as the necessary window size that would induce unrealistic tails in the data. Step changes in pH are due to dilutions. FIGS. 18B, 19B, and 20B also depict the duration of time, represented by the bars, for the culture solution to change a pH value by 0.1.

### Example—Optical Density

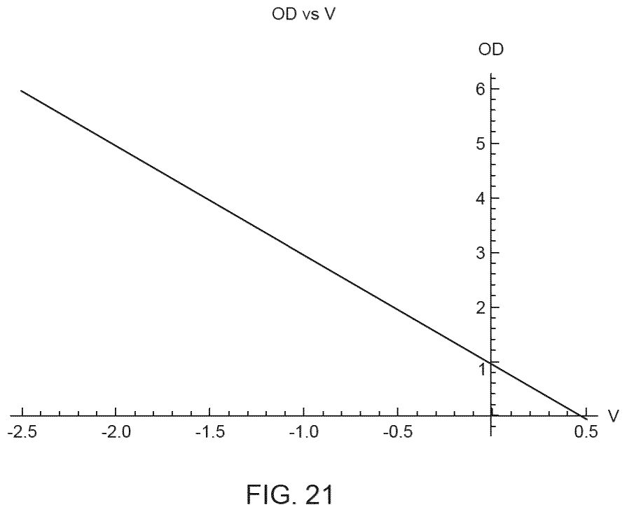

In one example, the system board is divided into 3 sections: The LED driver, TEC driver, and absorbance measurement circuit. The LED driver powers and controls the 970 nm wavelength LED to approximately 60% of full current, as greater intensity results in diminishing returns in this setup. LED ripple regulation is kept to <2% and is run in analog fashion rather than PWM to reduce constraints on timing. The TEC driver regulates the temperature of the photodiode mounting block to maintain both photodiodes at similar temperatures slightly below ambient. This helps match outputs of the photodiodes for consistent measurements across varying ambient conditions and reduces dark current. The absorbance measurement takes the log ratio of the sample photodiode signal against the reference photodiode signal. The output voltage of the log ratio amplifier is filtered, split into differential signals, and read by a 12-bit ADC. This measurement setup yields a span of about 0-6 OD at 0.0012 OD resolution, with estimated error at about 0.008 OD, as shown in FIG. 21.

The term “about,” “approximately,” or the symbol “˜” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, it may mean within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” or the symbol “˜” precedes a first numerical value in a series of two or more numerical values, it is understood that the term or symbol applies to each one of the numerical values in that series.

Other examples and implementations are within the scope and spirit of the disclosure and appended claims. For example, features implementing functions may also be physically located at various positions, including being distributed such that portions of functions are implemented at different physical locations. Also, as used herein, including in the claims, “or” as used in a list of items prefaced by “at least one of” indicates a disjunctive list such that, for example, a list of “at least one of A, B, or C” means A or B or C or AB or AC or BC or ABC (i.e., A and Band C). Further, the term “exemplary” does not mean that the described example is preferred or better than other examples.

The foregoing description, for purposes of explanation, uses specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A system configured to replicate natural conditions for a microorganism comprising:
a main enclosure comprising:
a lighting control module including a light source,
a pH control system,
plumbing defining a pathway between inlet valving and outlet valving,
one or more sensors selected from one or more of:
a pH sensor, a temperature sensor, a mass sensor, a light intensity sensor, a pressure sensor, volume sensor, optical density sensor, or a flow sensor,
a growth station platform configured to receive a growth chamber and comprising:
a weighing module to determine a mass of the growth chamber, and
a stir plate configured to stir a growth chamber, and
a temperature control to cool one or both of the main enclosure and the growth chamber.

2. The system of claim 1, further comprising:
a growth chamber defining an internal volume and having a non-reflective surface and configured to retain a culture solution; and
a cap defining a plurality of connections and selectively connected to a top of the growth chamber to selectively seal the internal volume;
wherein at least one of the one or more sensors is coupled to the cap and captures information about the culture solution.

3. The system of claim 2, wherein the growth chamber has a rectangular cross section corresponding to a voxel of a natural body of water.

4. The system of claim 2, wherein:
the plumbing is operatively connected with one or more inlet solution containers and the growth chamber at the cap, and
the inlet solution containers include one or more of media for the culture solution, dilution solution, a rinsing solution, or a cleaning solution.

5. The system of claim 2, wherein the light source is positioned at a top of the growth chamber to direct light vertically through the growth chamber.

6. The system of claim 2, wherein the pH control system includes a sparger to release a gas through the culture solution to raise or lower the pH value of the culture solution.

7. The system of claim 6, wherein the gas is one of carbon dioxide to decrease the pH value or air to increase pH value.

8. The system of claim 1, wherein the one or more sensors include an optical density sensor.

9. The system of claim 1, wherein the plumbing defines an at least mostly metal-free pathway for solutions of the system.

10. The system of claim 1, wherein the plumbing includes one or more peristaltic pumps to limit contamination of a solution of the system.

11. The system of claim 1, wherein the temperature control includes a thermoelectric cooler positioned at the growth chamber platform.

12. The system of claim 1, wherein the temperature control module includes a cooling system for the light source to limit radiant heat transmission from the light source towards the growth chamber platform.

13. The system of claim 1, further comprising:
a stackable electronics board assembly including at least one processing element, and
wherein the at least one processing element generates or executes a schedule to control one or more of a pH, temperature, liquid volume, dilution, or light intensity of the culture vessel.

14. The system of claim 13, wherein the at least one processing element is in operative communication with a remote device to selectively update a schedule or communicate information captured by the one or more sensors of the culture solution.

15. A system configured to control microorganism culture conditions comprising:
a culture vessel including a culture solution, wherein:
the culture vessel provides a liquid path at least partially free of contact with metal parts,
includes a modular cap designed to selectively connect with analytical probes or sensors,
the culture vessel has a square profile and defines a volume between 100 mL and one liter,
the culture vessel defines an at least partially non-reflective interior surface, and
the culture vessel includes a magnetic stirrer;
a lighting control module, including a lighting element selectively producing a stable light intensity range;
a temperature control module including a thermoelectric cooler positioned at a bottom of the culture vessel;
a pH control module operatively associated with a gas sparging control module to control the pH of the culture solution;
a liquid storage system including storage for one or more of:
input media, input dilution solution, input cleaning solution, waste solution, sampling solution;
an electronic system configured to execute a scheduling system, wherein the scheduling system defines events and set points for environmental parameters and liquid manipulations; and
a weighing module, wherein a mass of the culture vessel is associated with a volume of the culture solution.

16. A method for culturing a microorganism comprising:
filling a culture vessel having a non-reflective surface with a culture solution to emulate a pond environment under natural conditions;
operatively coupling the culture vessel to one or more of a weigh module, temperature control module, light source, plumbing, or a pH control module;
executing by a processing element, a schedule to control one or more of a pH, temperature, liquid volume, dilution, or light intensity of the culture vessel; and
maintaining the schedule to define culture vessel environment similar to outdoor ponds.

17. The method of claim 16, wherein the maintaining includes;
determining a pH of the culture solution by one or more sensors;
comparing the determined pH to a desired pH value; and
sparging one of air or carbon dioxide within the culture solution to increase or decrease the determined pH to approximate the desired pH value of the culture solution.

18. The method of claim 16, wherein the maintaining includes
determining a mass of the culture vessel by one or more sensors, wherein the mass is associated with a volume of the culture solution, and
adding an inlet solution to the culture vessel by the plumbing thereby increasing the volume of the culture solution.

19. The method of claim 16, wherein the maintaining includes:
removing a volume of the culture solution for sampling or waste; and
adding an inlet solution to the culture vessel by the plumbing to substantially replace the removed volume.

20. The method of claim 16, wherein:
the schedule defines the light intensity within the culture vessel for a period of a day, the light intensity corresponding to a specific geographical location, and
wherein the maintaining includes:
determining by one or more sensors a temperature of the culture solution. and
cooling the culture vessel by a thermoelectric cooler to adjust the temperature to a temperature corresponding to the light intensity and geographical location.
